(19) United States
(12) Patent Application Publication
(10) Pub. No.: US 2017/0251963 A1
(43) Pub. Date: Sep. 7, 2017

(54) MEASUREMENT APPARATUS AND DETECTION DEVICE

(71) Applicant: SEIKO EPSON CORPORATION, Tokyo (JP)

(72) Inventors: Taki HASHIMOTO, Shiojiri-shi (JP); Ayae SAWADO, Kai-shi (JP)

(73) Assignee: SEIKO EPSON CORPORATION, Tokyo (JP)

(21) Appl. No.: 15/437,724

(22) Filed: Feb. 21, 2017

(30) Foreign Application Priority Data
Mar. 4, 2016 (JP) 2016-042293

(51) Int. Cl. A61B 5/1455; A61B 5/00

(52) U.S. Cl. CPC A61B 5/14552; A61B 5/681; A61B 2562/0238; A61B 2562/0242; A61B 2562/043

(57) ABSTRACT

A measurement apparatus includes a first light emission unit that emits light having a first wavelength, a second light emission unit that emits light having a second wavelength of which an arrival depth with respect to a measurement site exceeds the arrival depth of the light having the first wavelength, a light reception unit that generates a detection signal corresponding to a light reception level of light arriving from the measurement site, and an analysis unit that acquires biological information corresponding to the detection signal. The first light emission unit, the second light emission unit, and the light reception unit are installed on a detection surface facing the measurement site, and a distance between the first light emission unit and the light reception unit exceeds a distance between the second light emission unit and the light reception unit.

### BACKGROUND

1. Technical Field

The present invention relates to a technology in which biological information is measured.

2. Related Art

In the related art, various types of measurement technologies in which biological information is measured in a non-invasive manner through light irradiation performed to a living body have been proposed. For example, JP-A-2006-75354 discloses a configuration in which light being emitted from a light emission window and being reflected inside a living body is received in each of a plurality of light reception windows, and a degree of oxygen saturation of the living body is measured based on a light reception result.

Incidentally, a depth inside a living body through which light arriving at a light reception point from a light emission point passes varies in accordance with a distance between the light emission point and the light reception point. As disclosed in JP-A-2006-75354, in a configuration in which distances between a light emission window and a plurality of light reception windows are different from each other, light emitted from the light emission window passes through the depths different from each other inside the living body and arrives at each of the plurality of light reception windows. Therefore, there is a problem in that biological information significantly varies in accordance with the type of tissue, the vascular density, and the like at a site inside the living body through which light arriving at each of the light reception units passes.

### SUMMARY

An advantage of some aspects of the invention is to measure biological information with high accuracy.

A measurement apparatus according to a favorable aspect of the invention includes a first light emission unit that emits light having a first wavelength, a second light emission unit that emits light having a second wavelength of which an arrival depth with respect to a measurement site exceeds the arrival depth of the light having the first wavelength, a light reception unit that generates a detection signal corresponding to a light reception level of light arriving from the measurement site, and an analysis unit that acquires biological information corresponding to the detection signal. The first light emission unit, the second light emission unit, and the light reception unit are installed on a detection surface facing the measurement site, and a distance between the first light emission unit and the light reception unit exceeds a distance between the second light emission unit and the light reception unit. When a distance between a light emission point and a light reception point increases, light tends to arrive at a position deep inside the measurement site. In the favorable aspect of the invention, based on the configuration in which the first light emission unit emits the light having the first wavelength and the second light emission unit emits the light having the second wavelength of which the arrival depth with respect to the measurement site exceeds the arrival depth of the light having the first wavelength, the distance between the first light emission unit and the light reception unit exceeds the distance between the second light emission unit and the light reception unit. Therefore, compared to a configuration in which the first light emission unit and the second light emission unit are positioned while being equidistant from the light reception unit, a propagation range of emission light from the first light emission unit and a propagation range of emission light from the second light emission unit inside the measurement site can approach or overlap each other in a depth direction of the measurement site. According to the configuration described above, compared to a configuration in which the propagation ranges deviate from each other between the emission light from the first light emission unit and the emission light from the second light emission unit, there is an advantage in that the biological information can be measured with high accuracy.

According to the favorable aspect of the invention, the first light emission unit, the second light emission unit, and the light reception unit are collinearly positioned. In the aspect described above, since the first light emission unit, the second light emission unit, and the light reception unit are collinearly positioned, for example, compared to a configuration in which the first light emission unit, the second light emission unit, and the light reception unit are not collinearly positioned, the propagation range of emission light from the first light emission unit and the propagation range of emission light from the second light emission unit can approach or overlap each other. Therefore, the above-described effect of being able to measure the biological information with high accuracy is particularly remarkable.

In the favorable aspect of the invention, the light reception unit may include a first light reception unit receiving light which is emitted from the first light emission unit and passes through the measurement site, and a second light reception unit receiving light which is emitted from the second light emission unit and passes through the measurement site, and a distance between the first light emission unit and the first light reception unit may exceed a distance between the second light emission unit and the second light reception unit. In the favorable aspect with this configuration, the distance between the first light emission unit and the first light reception unit exceeds the distance between the second light emission unit and the second light reception unit. Therefore, compared to a configuration in which the distance between the first light emission unit and the first light reception unit is equal to the distance between the second light emission unit and the second light reception unit, the propagation range of light arriving at the first light reception unit from the first light emission unit, and the propagation range of light arriving at the second light reception unit from the second light emission unit can approach or overlap each other in the depth direction of the measurement site. According to the configuration described above, compared to a configuration in which the propagation ranges deviate from each other between the emission light from the first light emission unit and the emission light from the second light emission unit, there is an advantage in that the biological information can be measured with high accuracy.

In the favorable aspect of the invention, the first light emission unit, the second light emission unit, the first light reception unit, and the second light reception unit may be collinearly positioned. In the favorable aspect with this configuration, since the first light emission unit, the second light emission unit, the first light reception unit, and the second light reception unit are collinearly positioned, the propagation range of light arriving at the first light reception unit from the first light emission unit, and the propagation range of light arriving at the second light reception unit from the second light emission unit can approach or overlap each other. Therefore, the above-described effect of being able to measure the biological information with high accuracy is particularly remarkable.

In the favorable aspect of the invention, the first light emission unit and the first light reception unit may be positioned between the second light emission unit and the second light reception unit. In the favorable aspect with this configuration, since a range in which emission light from the first light emission unit is propagated and a range in which emission light from the second light emission unit is propagated can sufficiently overlap each other, an error of the biological information caused due to the difference between the propagation ranges can be sufficiently restrained.

In the favorable aspect of the invention, a straight line passing through the first light emission unit and the first light reception unit and a straight line passing through the second light emission unit and the second light reception unit may intersect each other. In the favorable aspect with this configuration, since the straight line passing through the first light emission unit and the first light reception unit and the straight line passing through the second light emission unit and the second light reception unit intersect each other, there is an advantage in that the first light emission unit and the first light reception unit, and the second light emission unit and the second light reception unit can be disposed on the detection surface while avoiding excessive approach or interference therebetween.

In the favorable aspect of the invention, the light having the first wavelength may be near infrared light, and the light having the second wavelength may be red light. In addition, in another aspect of the invention, the light having the first wavelength may be green light, and the light having the second wavelength may be near infrared light or red light. However, the first wavelength and the second wavelength are not limited to the exemplification described above.

A detection device according to a favorable aspect of the invention generates a detection signal used for generating biological information. The detection device includes a first light emission unit that emits light having a first wavelength, a second light emission unit that emits light having a second wavelength of which an arrival depth with respect to a measurement site exceeds the arrival depth of the light having the first wavelength, and a light reception unit that generates a detection signal corresponding to a light reception level of light arriving from the measurement site. The first light emission unit, the second light emission unit, and the light reception unit are installed on a detection surface facing the measurement site, and a distance between the first light emission unit and the light reception unit exceeds a distance between the second light emission unit and the light reception unit.

### BRIEF DESCRIPTION OF THE DRAWINGS

The invention will be described with reference to the accompanying drawings, wherein like numbers reference like elements.

FIG. 1 is a side view of a measurement apparatus according to a first embodiment of the invention.

FIG. 2 is a configuration diagram focusing on a function of the measurement apparatus.

FIG. 3 is a view describing a relationship between a light emission-to-light reception distance and an arrival depth.

FIG. 4 is a graph describing a relationship between the light emission-to-light reception distance and the arrival depth.

FIG. 5 is a view describing a positional relationship between a light emission unit and a light reception unit.

FIG. 6 is a view describing a positional relationship between the light emission unit and the light reception unit in a second embodiment.

FIG. 7 is a view describing a positional relationship between the light emission unit and the light reception unit in a third embodiment.

FIG. 8 is a view describing a positional relationship between the light emission unit and the light reception unit in a modification example of the third embodiment.

FIG. 9 is a configuration diagram of a measurement apparatus according to a fourth embodiment.

FIG. 10 is a configuration diagram of a measurement apparatus according to a modification example of the fourth embodiment.

### DESCRIPTION OF EXEMPLARY EMBODIMENTS

### First Embodiment

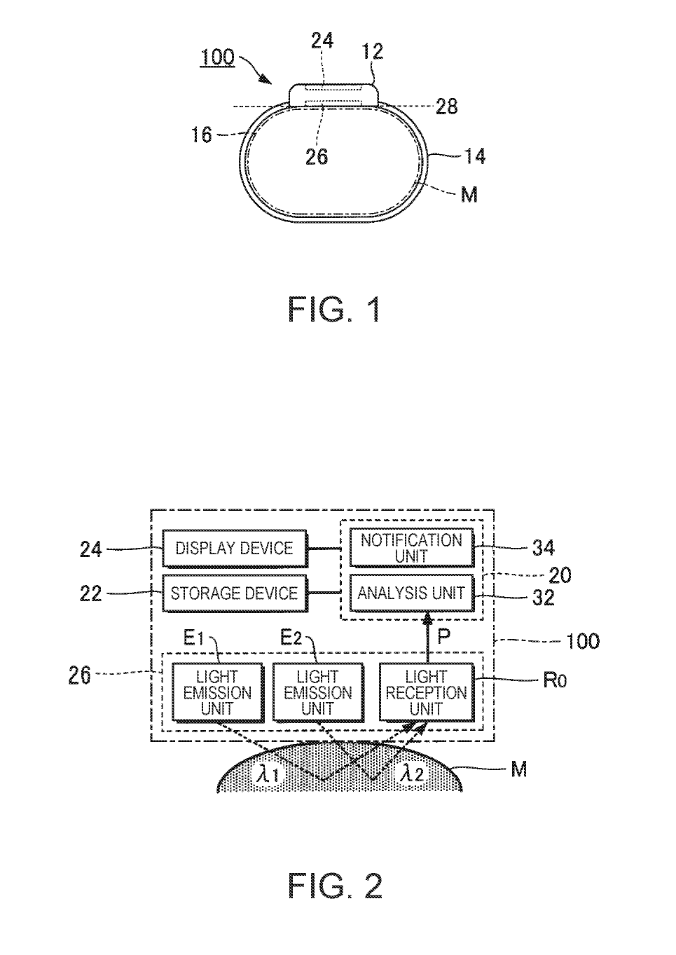

FIG. 1 is a side view of a measurement apparatus 100 according to a first embodiment of the invention. The measurement apparatus 100 of the first embodiment is a biological measurement instrument which measures biological information of a test subject in a non-invasive manner. The measurement apparatus 100 is mounted on a site (hereinafter, will be referred to as “measurement site”) M which becomes a measurement target in the body of the test subject. The measurement apparatus 100 of the first embodiment is portable wristwatch-type equipment provided with a housing unit 12 and a belt 14. The measurement apparatus 100 can be mounted on a wrist of the test subject when the belt 14 is wound around the wrist which is an exemplification of the measurement site M. The measurement apparatus 100 of the first embodiment comes into contact with a surface 16 of the wrist of the test subject. In the first embodiment, the degree of oxygen saturation (SpO2) is exemplified as the biological information. The degree of oxygen saturation denotes a ratio (%) of oxygen-binding hemoglobin to hemoglobin in blood of the test subject. The degree of oxygen saturation is an index for evaluating a respiratory function of the test subject.

FIG. 2 is a configuration diagram focusing on a function of the measurement apparatus 100. As exemplified in FIG. 2, the measurement apparatus 100 of the first embodiment is provided with a control device 20, a storage device 22, a display device 24, and a detection device 26. The control device 20 and the storage device 22 are installed inside the housing unit 12. As exemplified in FIG. 1, the display device 24 (for example, liquid crystal display panel) is installed on a surface (for example, a surface on a side opposite to the measurement site M) of the housing unit 12. The display device 24 displays various types of images including measurement results in response to controlling of the control device 20.

The detection device 26 in FIG. 2 is a sensor module which generates a detection signal P corresponding to the state of the measurement site M. For example, the detection device 26 is installed on a surface (hereinafter, will be referred to as “detection surface”) 28 facing the measurement site M in the housing unit 12. The detection surface 28 is a flat surface or a curved surface. As exemplified in FIG. 2, the detection device 26 of the first embodiment is provided with a light emission unit E1, a light emission unit E2, and a light reception unit R0. The light emission unit E1, the light emission unit E2, and the light reception unit R0 are installed on the detection surface 28 and are positioned on one side when viewed from the measurement site M.

For example, each of the light emission unit E1 and the light emission unit E2 is configured to include a light emitting element such as a light emitting diode (LED). The light emission unit E1 (exemplification of first light emission unit) is a light source which emits light having a wavelength λ1 to the measurement site M. The light emission unit E2 (exemplification of second light emission unit) is a light source which emits light having a wavelength λ2 different from the wavelength λ1 to the measurement site M. In the first embodiment, for convenience, a case where the light emission unit E1 emits near infrared light (λ1=900 nm) and the light emission unit E2 emits red light (λ2=700 nm) is postulated. The wavelength λ1 and the wavelength λ2 are not limited to the exemplification described above. For example, the wavelength λ1 can be set to 940 nm and the wavelength λ2 can be set to 660 nm.

Emission light from each of the light emission unit E1 and the light emission unit E2 is incident on the measurement site M and is repetitively reflected and diffused inside the measurement site M. Thereafter, the emission light is emitted toward the detection surface 28 side and arrives at the light reception unit R0. In other words, the detection device 26 of the first embodiment is a reflection-type optical sensor. The light reception unit R0 generates the detection signal P corresponding to a light reception level of light arriving from the measurement site M. For example, a photoelectric transducer such as a photo diode (PD) which receives light with a reception surface facing the measurement site M is favorably utilized as the light reception unit R0. A blood vessel in the measurement site M iteratively expands and contracts at a cycle equal to that of heartbeat. Since the quantities of light absorbed by blood inside a blood vessel are different from each other between an expansion phase and a contraction phase, the detection signal P generated by the light reception unit R0 so as to correspond to the light reception level of light from the measurement site M is a pulse wave signal including a cyclical variation component matching a pulsation component (volume pulse wave) of the artery of the measurement site M. For example, the detection device 26 includes a drive circuit which drives the light emission unit E1 and the light emission unit E2 with a supplied driving current, and output circuits (for example, an amplification circuit and an AD converter) for amplifying and AD-converting an output signal of the light reception unit R0. However, each of the circuits is not illustrated in FIG. 1.

The control device 20 in FIG. 2 is an arithmetic processing device such as a central processing unit (CPU) and a field-programmable gate array (FPGA). The control device 20 controls the measurement apparatus 100 in its entirety. For example, the storage device 22 is configured with a nonvolatile semiconductor memory and stores a program which is executed by the control device 20 or various types of data used by the control device 20. The control device 20 of the first embodiment executes the program stored in the storage device 22, thereby realizing a plurality of functions (analysis unit 32 and notification unit 34) for measuring the degree of oxygen saturation of the test subject. It is possible to employ a configuration in which the functions of the control device 20 are distributed in a plurality of integrated circuits, or a configuration in which a part or all of the functions of the control device 20 are realized through a dedicated electronic circuit. In addition, in FIG. 2, the control device 20 and the storage device 22 are illustrated as separate elements. However, the control device 20 internally equipped with the storage device 22 can be realized through an application specific integrated circuit (ASIC), for example.

The analysis unit 32 specifies a degree S of oxygen saturation of the test subject based on the detection signal P generated by the detection device 26. The notification unit 34 causes the display device 24 to display the degree S of oxygen saturation specified by the analysis unit 32. It is favorable to provide a configuration in which the notification unit 34 notifies a user of warning (possibility of failure of the respiratory function) in a case where the degree S of oxygen saturation varies to a numerical value beyond a predetermined range.

When the degree S of oxygen saturation is specified by the analysis unit 32, a known technology can be arbitrarily employed. For example, the degree S of oxygen saturation can be specified by utilizing the correspondence between a variation ratio Φ calculated based on the detection signal P, and the degree S of oxygen saturation. As expressed through the following Mathematical Expression (1), the variation ratio Φ is a rate of a component ratio C2 with respect to a component ratio C1. The component ratio C1 is an intensity ratio of a steady component Q1 (DC) to a variation component Q1 (AC) of the detection signal P when the light emission unit E1 emits the light having the wavelength λ1. The component ratio C2 is an intensity ratio of a steady component Q2 (DC) to a variation component Q2 (AC) of the detection signal P when the light emission unit E2 emits the light having the wavelength λ2. The variation component Q1 (AC) and the variation component Q2 (AC) are components which are interlocked with pulsations of the artery of the test subject and cyclically vary (pulse wave components). The steady component Q1 (DC) and the steady component Q2 (DC) are temporal components which are regularly maintained. The variation ratio Φ and the degree S of oxygen saturation in Mathematical Expression (1) are correlated to each other.

$$Φ = \frac{{C}_{2}}{{C}_{1}} = \frac{{Q}_{2 ( AC )} / {Q}_{2 ( DC )}}{{Q}_{1 ( AC )} / {Q}_{1 ( DC )}} \qquad ( 1 )$$

The analysis unit 32 extracts the variation component Q1 (AC) and the steady component Q1 (DC), and the variation component Q2 (AC) and the steady component Q2 (DC) through an analysis of the detection signal P at the time the light emission unit E1 and the light emission unit E2 are alternately emitted in a sufficiently short cycle compared to the pulse, thereby calculating the variation ratio Φ. The analysis unit 32 refers to a table in which each of the numerical values of the variation ratio Φ and each of the numerical values of the degree S of oxygen saturation correspond to each other, thereby specifying the degree S of oxygen saturation corresponding to the variation ratio Φ calculated based on the detection signal P, as a measurement result.

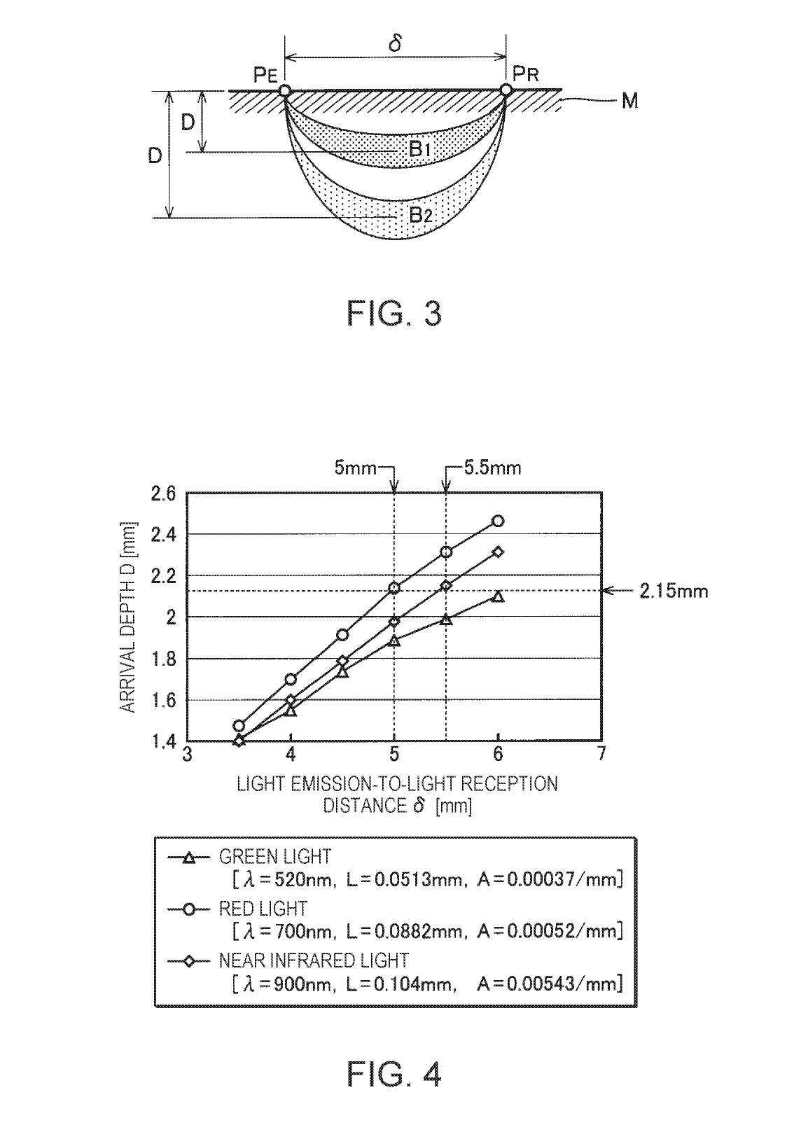

As exemplified in FIG. 3, a condition in which light which is emitted from an arbitrary light emission point PE and passes through the inside of the measurement site M is received at a light reception point PR is postulated. FIG. 4 shows a simulation result of light propagation inside the measurement site M of FIG. 3. In FIG. 4, a relationship between a distance δ from the light emission point PE to the light reception point PR (hereinafter, will be referred to as “light emission-to-light reception distance”) and a depth (distance from the surface of a living body) D at which light arrives inside the measurement site M is illustrated for each of green light (wavelength λ=520 nm), red light (wavelength λ=700 nm), and near infrared light (wavelength λ=900 nm). The simulation of light propagation is conducted through the Monte Carlo method employing conditions in which there is no loss in a diffusion phenomenon and light is attenuated between the diffusion phenomena due to the Lambert-Beer law. A free path L and an absorption coefficient A of the diffusion are set to numerical values in FIG. 4 postulated for the derma of a living body. The depth D in FIG. 4 denotes the most frequent depth at which photons arriving at the light reception point PR from the light emission point PE pass through the inside of the measurement site M. Specifically, as expressed through the following Mathematical Expression (2), a representative depth D can be calculated by weighting a depth l with a weighted value W corresponding to the number of photons within a virtual vertical section which is set between the light emission point PE and the light reception point PR. The character z in Mathematical Expression (2) denotes a coordinate axis parallel to a depth direction of the measurement site M.

$$D = \frac{\int W ldz}{\int Wdz} \qquad ( 2 )$$

As it is understood from FIG. 4, degrees of light which is incident on the measurement site M from the light emission point PE and arrives at a position deep inside the measurement site M (hereinafter, will be referred to as “arrival depth”) are different from each other in accordance with the wavelength λ. Specifically, the arrival depth of green light tends to fall below the arrival depth of near infrared light, and the arrival depth of red light tends to exceed the arrival depth of near infrared light. In other words, near infrared light is likely to arrive at a deep portion inside the measurement site M compared to green light, and red light is likely to arrive at a deep portion inside the measurement site M compared to near infrared light or green light. For example, on the postulation of a case where the light emission-to-light reception distance δ is 6 mm, near infrared light arrives at the depth D of 2.31 mm from the surface of the measurement site M. In contrast, red light arrives at the depth D of 2.45 mm from the surface of the measurement site M. As it is understood from the description above, in the first embodiment, the arrival depth of red light (λ2=700 nm) emitted from the light emission unit E2 exceeds the arrival depth of near infrared light (λ1=900 nm) emitted from the light emission unit E1.

As described above, since the arrival depth depends on the wavelength λ, in a case where rays of light having the wavelengths λ different from each other are emitted from the light emission point PE under the condition of the common light emission-to-light reception distance δ, as exemplified in FIG. 3, the depth of a range (hereinafter, will be referred to as “propagation range”) B in which light arriving at the light reception point PR from the light emission point PE is propagated inside the measurement site M varies in accordance with the wavelength λ. The propagation range B denotes a range in which light having the intensity exceeding a predetermined value is distributed (so-called a banana shape).

For example, in a configuration in which the light emission unit E1 and the light emission unit E2 are respectively installed at the light emission points PE equidistant from the light reception point PR where the light reception unit R0 is installed (hereinafter, will be referred to as “comparative example”), as exemplified in FIG. 3, a propagation range B1 of emission light from the light emission unit E1 and a propagation range B2 of emission light from the light emission unit E2 are different from each other in depth. Specifically, the propagation range B2 of red light emitted by the light emission unit E2 is distributed at a deep position compared to the propagation range B1 of near infrared light emitted by the light emission unit E1. In other words, in a configuration of the comparative example, rays of emission light from the light emission unit E1 and the light emission unit E2 respectively pass through sites (depths) different from each other for each of the wavelengths λ and arrive at the light reception unit R0 inside the measurement site M.

As exemplified above, under the condition in which the propagation ranges B of the emission light deviate from each other between the light emission unit E1 and the light emission unit E2, the types of tissue inside the measurement site M (for example, epiderm and derma), the degrees of vascular density, and the like are different from each other between a site through which emission light of the light emission unit E1 and a site through which emission light of the light emission unit E2. Therefore, the optical characteristics such as the absorbance and the concentration can also be different from each other, thereby leading to a problem of a significant error of the degree S of oxygen saturation. In consideration of the above-described circumstances, in the first embodiment, positions of the light emission unit E1, the light emission unit E2, and the light reception unit R0 are selected such that the depth D at which light having the wavelength λ1 and being emitted by the light emission unit E1 arrives and the depth D at which light having the wavelength λ2 and being emitted by the light emission unit E2 approach each other.

As it is understood from FIG. 4, when the light emission-to-light reception distance δ becomes significant, the depth D at which light arrives inside the measurement site M tends to increase (arrives at a deeper position). In consideration of the above-described tendency, in the first embodiment, positions of the light emission unit E1, the light emission unit E2, and the light reception unit R0 are selected such that light having a smaller arrival depth (light unlikely to arrive at a position deep inside the measurement site M) is emitted from a position farther from the light reception unit R0.

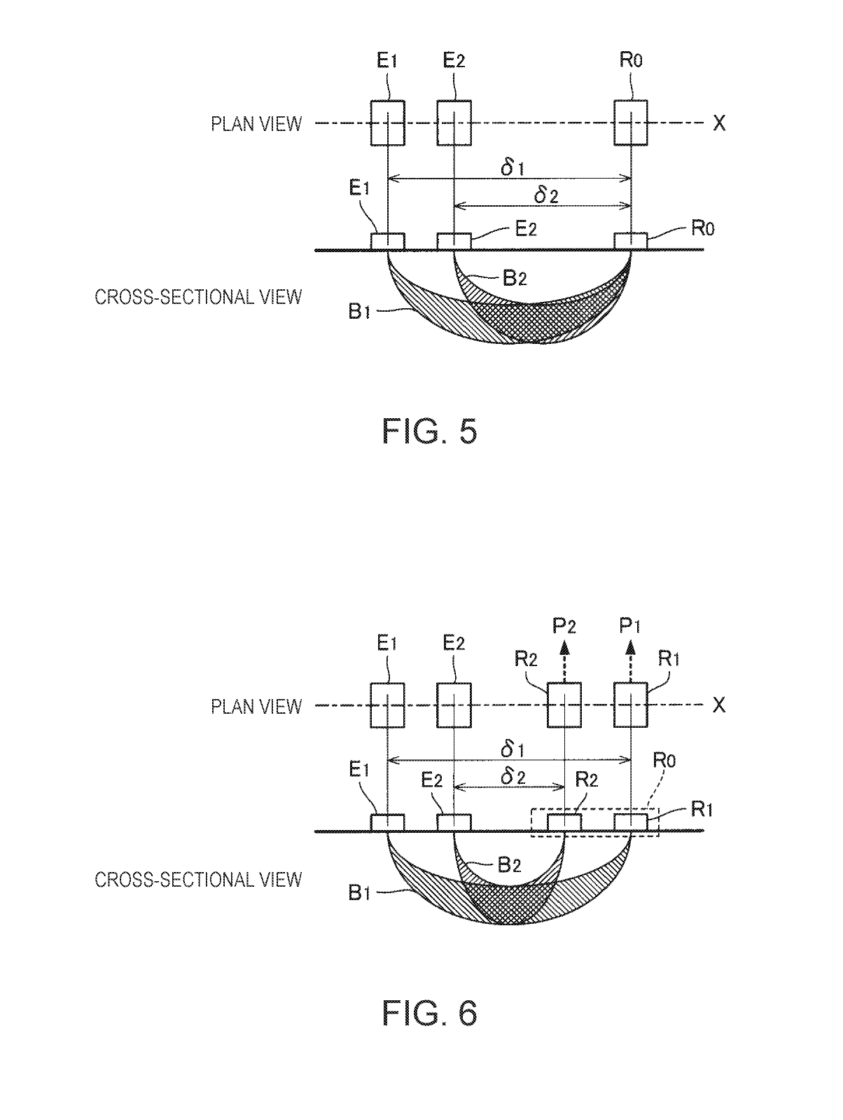

FIG. 5 is a plan view and a cross-sectional view exemplifying a positional relationship among the light emission unit E1, the light emission unit E2, and the light reception unit R0. As described above, in the first embodiment, the arrival depth of red light emitted from the light emission unit E2 exceeds the arrival depth of near infrared light emitted from the light emission unit E1. Therefore, as exemplified in FIG. 5, positions of the light emission unit E1, the light emission unit E2, and the light reception unit R0 are selected such that a distance δ1 between the light emission unit E1 and the light reception unit R0 exceeds a distance δ2 between the light emission unit E2 and the light reception unit R0 (δ1>δ2).

As exemplified in FIG. 5, the light emission unit E1, the light emission unit E2, and the light reception unit R0 are positioned on a straight line X on the detection surface 28 in a planar view (that is, when viewed in a direction perpendicular to the detection surface 28). Specifically, the centers of the light emission unit E1, the light emission unit E2, and the light reception unit R0 are positioned on the straight line X. In the first embodiment, the light emission unit E1 is positioned on a side opposite to the light reception unit R0 so as to interpose the light emission unit E2 therebetween. In other words, the configuration can also be mentioned as a configuration in which the light emission unit E2 is positioned on the straight line X connecting the light emission unit E1 and the light reception unit R0, or a configuration in which the light emission unit E1, the light emission unit E2, and the light reception unit R0 are collinearly arrayed. As a result in which the above-described configuration is employed, in the first embodiment, as exemplified in FIG. 5, the propagation range B1 of near infrared light emitted from the light emission unit E1 and the propagation range B2 of red light emitted from the light emission unit E2 overlap each other.

For example, as exemplified in FIG. 4, in a case where rays of light of both the light emission unit E1 and the light emission unit E2 pass through the depth D of 2.15 mm from the surface of the measurement site M, the light emission unit E1 is disposed at a position separated from the light reception unit R0 as much as the distance δ1 of approximately 5.5 mm, and the light emission unit E2 is disposed at a position separated from the light reception unit R0 as much as the distance δ2 of approximately 5 mm. The distance between the light emission unit E1 and the light emission unit E2 (for example, distance between the centers) is set within a range from 300 μm to 500 μm, for example.

As described above, in the first embodiment, based on the configuration in which the light emission unit E1 emits near infrared light having the wavelength λ1 (exemplification of first wavelength) and the light emission unit E2 emits red light having the wavelength λ2 (exemplification of second wavelength) of which the arrival depth with respect to the measurement site M exceeds the arrival depth of the near infrared light, the distance δ1 between the light emission unit E1 and the light reception unit R0 exceeds the distance δ2 between the light emission unit E2 and the light reception unit R0. Therefore, compared to the comparative example in which the light emission unit E1 and the light emission unit E2 are positioned while being equidistant from the light reception unit R0, as exemplified in FIG. 5, the propagation range B1 of near infrared light emitted by the light emission unit E1 and the propagation range B2 of red light emitted by the light emission unit E2 can approach or overlap each other. In the configuration described above, compared to a configuration in which the propagation ranges B (B1 and B2) deviate from each other between the emission light from the light emission unit E1 and the emission light from the light emission unit E2, the types of tissue inside the measurement site M, the degrees of vascular density, and the like approximate to each other between the propagation range B1 of emission light of the light emission unit E1 and the propagation range B2 of emission light of the light emission unit E2. Therefore, the optical characteristics such as the absorbance and the concentration can also approximate to each other. Therefore, there is an advantage in that an error caused due to the difference between the propagation ranges B can be restrained and the degree S of oxygen saturation can be specified with high accuracy.

In addition, in the first embodiment, the light emission unit E1, the light emission unit E2, and the light reception unit R0 are positioned on the straight line X. Therefore, compared to a configuration in which the light emission unit E1, the light emission unit E2, and the light reception unit R0 are not collinearly positioned, the propagation range B1 of emission light from the light emission unit E1 and the propagation range B2 of emission light from the light emission unit E2 can sufficiently approach or overlap each other. Therefore, the above-described effect of being able to specify the degree S of oxygen saturation with high accuracy is particularly remarkable.

Incidentally, as exemplified in the first embodiment, an error of the degree S of oxygen saturation caused due to the difference between the propagation ranges B has become a disadvantage apparent in the reflection-type optical sensor in which the light emission unit E1, the light emission unit E2, and the light reception unit R0 are positioned on one side with respect to the measurement site M. On the other hand, in a transmissive optical sensor in which the light emission unit E1 and the light emission unit E2 are positioned on a side opposite to the light reception unit R0 so as to interpose the measurement site M therebetween, emission light from the light emission unit E1 and emission light from the light emission unit E2 are propagated through paths approaching each other inside the measurement site M, thereby arriving at the light reception unit R0. Therefore, an error of the degree S of oxygen saturation caused due to the difference between the propagation ranges B becomes no particular problem. In consideration of the circumstances described above, it is possible to mention that the configuration in which the distance δ1 between the light emission unit E1 and the light reception unit R0 exceeds the distance δ2 between the light emission unit E2 and the light reception unit R0 is particularly effective for the reflection-type optical sensor.

### Second Embodiment

A second embodiment of the invention will be described. In each of the configurations exemplified below, the reference sign used in the description of the first embodiment will be applied to the element having the operation or the function similar to that of the first embodiment, and the detailed description thereof will be suitably omitted.

FIG. 6 is a plan view and a cross-sectional view exemplifying a positional relationship among the light emission unit E1, the light emission unit E2, and the light reception unit R0 in the second embodiment. As exemplified in FIG. 6, the light reception unit R0 of the second embodiment includes a light reception unit R1 (exemplification of first light reception unit) and a light reception unit R2 (exemplification of second light reception unit) which are installed on the detection surface 28. The light reception unit R1 and the light reception unit R2 are photoelectric transducers such as photo diodes receiving light with the reception surface facing the measurement site M. The light reception unit R1 receives near infrared light (wavelength λ1) which is emitted from the light emission unit E1 and passes through the measurement site M, thereby generating a detection signal P1 corresponding to the light reception level. The light reception unit R2 receives red light (wavelength λ2) which is emitted from the light emission unit E2 and passes through the measurement site M, thereby generating a detection signal P2 corresponding to the light reception level. The analysis unit 32 calculates the component ratio C1 of the above-referenced Mathematical Expression (1) based on the detection signal P1 generated by the light reception unit R1 and calculates the component ratio C2 of Mathematical Expression (1) based on the detection signal P2 generated by the light reception unit R2. The configuration and the method in which the analysis unit 32 specifies the degree S of oxygen saturation based on the variation ratio Φ of the component ratio C1 to the component ratio C2 are similar to those of the first embodiment.

As exemplified in FIG. 6, the light emission unit E1, the light emission unit E2, the light reception unit R1, and the light reception unit R2 are positioned on the straight line X on the detection surface 28 in a planar view. The distance δ1 between the light emission unit E1 and the light reception unit R1 exceeds the distance δ2 between the light emission unit E2 and the light reception unit R2 (δ1>δ2). Specifically, the light emission unit E2 and the light reception unit R2 are positioned between the light emission unit E1 and the light reception unit R1.

As described above, in the second embodiment, based on the configuration in which the light emission unit E1 emits near infrared light having the wavelength λ1 and the light emission unit E2 emits red light having the wavelength λ2, the distance δ1 between the light emission unit E1 and the light reception unit R1 exceeds the distance δ2 between the light emission unit E2 and the light reception unit R2. In the configuration described above, as exemplified in FIG. 6, the propagation range B1 of emission light from the light emission unit E1 and the propagation range B2 of emission light from the light emission unit E2 approach or overlap each other. Therefore, similar to the first embodiment, there is an advantage in that an error caused due to the difference between the propagation ranges B of the light emission unit E1 and the light emission unit E2 can be restrained and the degree S of oxygen saturation can be specified with high accuracy.

Particularly in the second embodiment, since the light emission unit E1, the light emission unit E2, the light reception unit R1, and the light reception unit R2 are positioned on the straight line X, the propagation range B1 and the propagation range B2 can sufficiently approach or overlap each other. Therefore, the above-described effect of being able to specify the degree S of oxygen saturation with high accuracy is particularly remarkable. Besides, in the second embodiment, since the light emission unit E2 and the light reception unit R2 are positioned between the light emission unit E1 and the light reception unit R1, an error of the degree S of oxygen saturation caused due to the difference between the propagation range B1 and the propagation ranges B2 can be sufficiently restrained.

### Third Embodiment

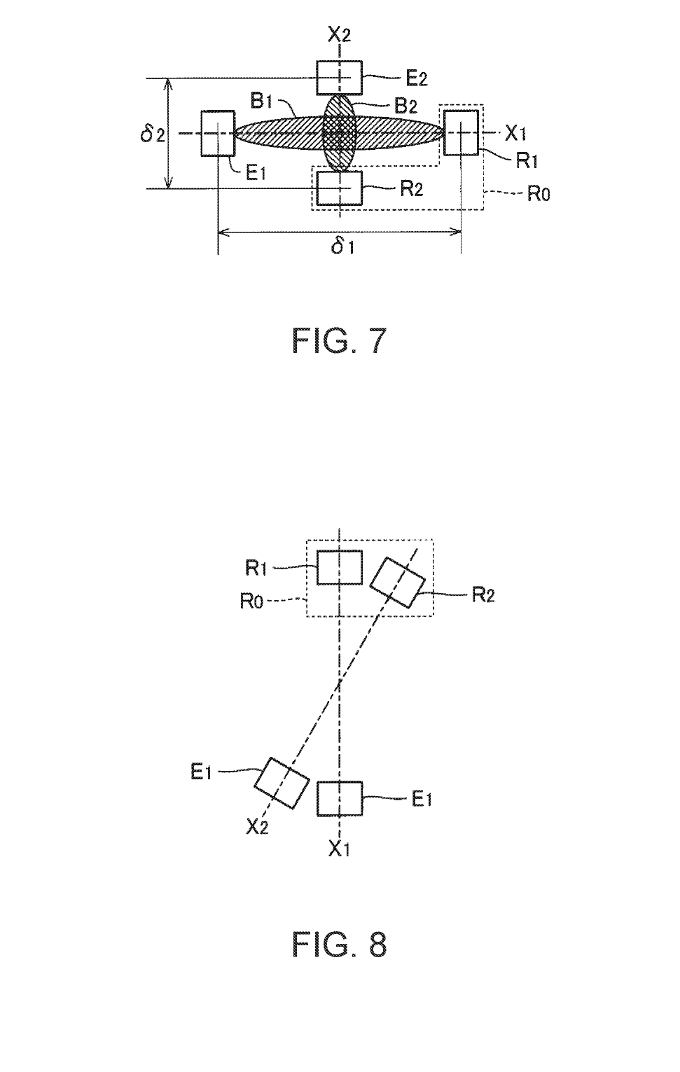

FIG. 7 is a plan view exemplifying a positional relationship among the light emission unit E1, the light emission unit E2, and the light reception unit R0 in a third embodiment. As exemplified in FIG. 7, similar to the second embodiment, the light reception unit R0 of the third embodiment includes the light reception unit R1 and the light reception unit R2. The light reception unit R1 receives near infrared light (wavelength λ1) which is emitted from the light emission unit E1 and passes through the measurement site M, thereby generating the detection signal P1 corresponding to the light reception level. The light reception unit R2 receives red light (wavelength λ2) which is emitted from the light emission unit E2 and passes through the measurement site M, thereby generating a detection signal P2 corresponding to the light reception level. The configuration and the method in which the analysis unit 32 specifies the degree S of oxygen saturation based on the detection signal P1 and the detection signal P2 are similar to those of the second embodiment.

As exemplified in FIG. 7, a straight line X1 passing through the light emission unit E1 and the light reception unit R1 and a straight line X2 passing through the light emission unit E2 and the light reception unit R2 intersect each other in a planar view. The straight line X1 passes through the center of the light emission unit E1 and the center of the light reception unit R1, and the straight line X2 passes through the center of the light emission unit E2 and the center of the light reception unit R2. As exemplified in FIG. 7, the straight line X1 and the straight line X2 are orthogonal to each other.

The straight line X1 intersects the straight line X2 at the middle point between the light emission unit E2 and the light reception unit R2. Similarly, the straight line X2 intersects the straight line X1 at the middle point between the light emission unit E1 and the light reception unit R1. The condition in which the distance δ1 between the light emission unit E1 and the light reception unit R1 exceeds the distance δ2 between the light emission unit E2 and the light reception unit R2 is similar to the first embodiment and the second embodiment. As it is understood from the description above, in the second embodiment, the light emission unit E1, the light emission unit E2, the light reception unit R1, and the light reception unit R2 are respectively positioned at rhombic apexes defined on the detection surface 28. According to the configuration described above, the propagation range B1 of emission light from the light emission unit E1 and the propagation range B2 of emission light from the light emission unit E2 approach or overlap each other below the intersection point of the straight line X1 and the straight line X2.

As described above, in the third embodiment as well, since the distance δ1 between the light emission unit E1 and the light reception unit R1 exceeds the distance δ2 between the light emission unit E2 and the light reception unit R2, the propagation range B1 of emission light from the light emission unit E1 and the propagation range B2 of emission light from the light emission unit E2 can approach or overlap each other. Therefore, similar to the second embodiment, there is an advantage in that an error caused due to the difference between the propagation ranges B of the light emission unit E1 and the light emission unit E2 can be restrained and the degree S of oxygen saturation can be specified with high accuracy. In addition, in the third embodiment, since the straight line X1 passing through the light emission unit E1 and the light reception unit R1 and the straight line X2 passing through the light emission unit E2 and the light reception unit R2 intersect each other, there is an advantage in that the light emission unit E1 and the light reception unit R1, and the light emission unit E2 and the light reception unit R2 can be disposed on the detection surface 28 while avoiding excessive approach or interference therebetween.

In FIG. 7, the configuration in which the straight line X1 and the straight line X2 orthogonal to each other is exemplified. However, the intersecting angle between the straight line X1 and the straight line X2 is not limited to the right angle. For example, as exemplified in FIG. 8, the light emission unit E1, the light reception unit R1, the light emission unit E2, and the light reception unit R2 can be disposed such that the straight line X1 and the straight line X2 intersect each other at a non-right angle. In the configuration of the third embodiment in which the straight line X1 and the straight line X2 intersect each other, it is favorable to adopt a configuration in which the distance δ1 between the light emission unit E1 and the light reception unit R1 exceeds the distance δ2 between the light emission unit E2 and the light reception unit R2. However, as exemplified in FIG. 8, it is possible to employ a configuration in which the straight line X1 and the straight line X2 intersect each other while the distance δ1 and the distance δ2 are distances equal to each other.

### Fourth Embodiment

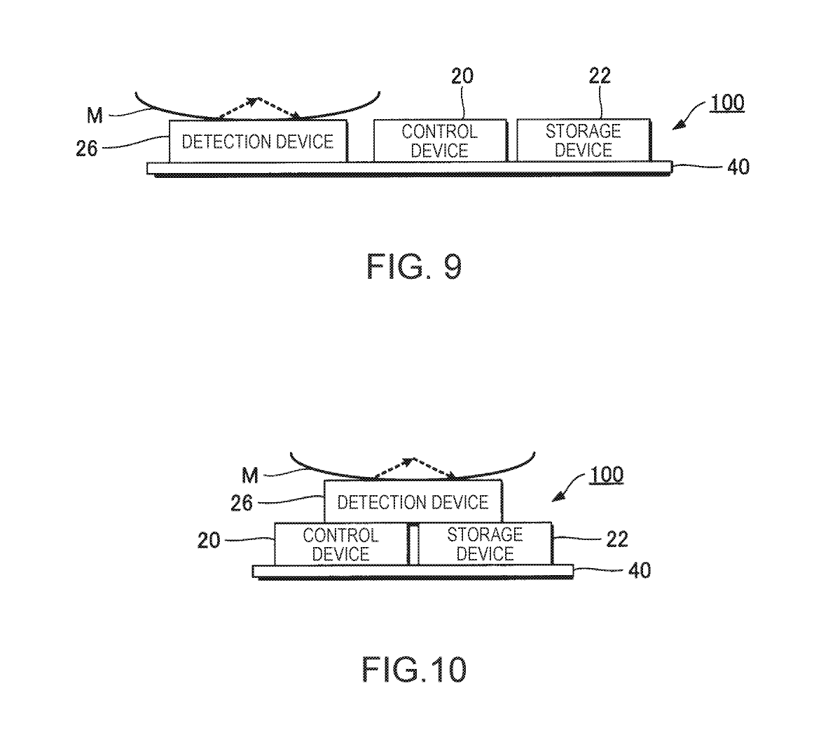

In each of the embodiments described above, the portable measurement apparatus 100 provided with the housing unit 12 and the belt 14 is exemplified. A measurement apparatus 100 of a fourth embodiment is a measurement module which does not include the housing unit 12 and the belt 14. Specifically, as exemplified in FIG. 9, the measurement apparatus 100 of the fourth embodiment is an electronic component configured to have the control device 20, the storage device 22, and the detection device 26 mounted on a substrate 40 (for example, circuit board). As exemplified in FIG. 10, it is favorable to have a configuration in which the control device 20 and the storage device 22 are mounted on the substrate 40 and the detection device 26 is disposed at a position close to the measurement site M compared to the control device 20 and the storage device 22. For example, portable equipment is configured by embedding the measurement apparatus 100 (measurement module) of the fourth embodiment in a housing in which the display device 24 is installed. The configuration and the function of each of the control device 20, the storage device 22, and the detection device 26 are similar to those of the embodiments described above. It is possible to realize a single body of the detection device 26 (portion not including the control device 20 and the storage device 22) through a form of the measurement module in which the housing unit 12, the belt 14, and the like are omitted.

### Modification Example

Each of the embodiments exemplified above can be variously modified. Specific modified aspects will be exemplified below. Two or more aspects arbitrarily selected from the exemplifications below can also be suitably combined together.

(1) In each of the embodiments described above, the configuration in which the light emission unit E1 emits near infrared light and the light emission unit E2 emits red light is exemplified. However, the wavelength λ of emission light emitted by the light emission unit E1 and the light emission unit E2 is not limited to the exemplification described above. For example, a configuration in which the light emission unit E1 emits green light (λ1=520 nm) and the light emission unit E2 emits near infrared light (λ2=900 nm) or red light (λ2=700 nm) can also be employed. As described with reference to FIG. 4, the arrival depth of green light falls below the arrival depths of near infrared light and red light. In other words, each of the configurations exemplified above is comprehensively expressed as a configuration in which the light emission unit E1 emits the light having the wavelength λ1, the light emission unit E2 emits light having the wavelength λ2 of which the arrival depth with respect to the measurement site M exceeds the arrival depth of the light having the wavelength λ1, and the distance δ1 between the light emission unit E1 and the light reception unit R0 exceeds the distance δ2 between the light emission unit E2 and the light reception unit R0.

(2) The degree S of oxygen saturation can also be arithmetically calculated. The calculation of the degree S of oxygen saturation performed by utilizing the detection signal P will be examined below. First, the Lambert-Beer expression related to optical attenuation is expressed through the following Mathematical Expression (3).

$$\{ ( 1 - S ) \cdot {E}_{d} + S \cdot {E}_{a} \} \cdot {C}_{a} \cdot Δ {I}_{a} = \frac{Δ {I}_{out}}{{I}_{out}} \qquad ( 3 )$$

The character Ed in Mathematical Expression (3) denotes the molar absorbance of deoxygenated hemoglobin, and the character Eo denotes the molar absorbance of oxygenated hemoglobin. The character Ca denotes the hemoglobin concentration, and the character Δla denotes the optical path length. The character ΔIout corresponds to the variation component Q1 (AC) or the variation component Q2 (AC) described above, and the character Iout corresponds to the steady component Q1 (DC) or the steady component Q2 (DC) described above. A ratio of a result in which variables (Q1 (AC), Q1 (DC)) related to light having the wavelength λ1 are applied to Mathematical Expression (1) to a result in which variables (Q2 (AC), Q2 (DC)) related to light having the wavelength λ2 are applied to Mathematical Expression (1) is expressed through the following Mathematical Expression (4). In Mathematical Expression (4), the reference sign λ1 is applied to an element related to the wavelength λ1, and the reference sign λ2 is applied to an element related to the wavelength λ2.

$$\begin{matrix}\frac{Δ {I}_{out} [ {λ}_{1} ] / {I}_{out} [ {λ}_{1} ]}{Δ {I}_{out} [ {λ}_{2} ] / {I}_{out} [ {λ}_{2} ]} = \frac{\{ ( 1 - S ) \cdot {E}_{d} [ {λ}_{2} ] + S \cdot {E}_{o} [ {λ}_{2} ] \} \cdot {C}_{a} \cdot Δ {l}_{a}}{\{ ( 1 - S ) \cdot {E}_{d} [ {λ}_{1} ] + S \cdot {E}_{o} [ {λ}_{1} ] \} \cdot {C}_{a} \cdot Δ {l}_{a}} \\ = \frac{{Q}_{2 ( AC )} / {Q}_{2 ( DC )}}{{Q}_{1 ( AC )} / {Q}_{1 ( DC )}} = Φ\end{matrix} \qquad ( 4 )$$

When it is assumed that the propagation range B1 of emission light from the light emission unit E1 and the propagation range B2 of emission light from the light emission unit E2 are common, the hemoglobin concentration Ca and the optical path length Δla in the numerator and the denominator on the right side in Mathematical Expression (4) are deleted. Therefore, the following Mathematical Expression (5) describing a relationship between the variation ratio Φ and the degree S of oxygen saturation is derived. Since the molar absorbance (Ed [λ1] and Ed [λ2]) of deoxygenated hemoglobin and the molar absorbance (Eo [λ1] and Eo [λ2]) of oxygenated hemoglobin are known, when the analysis unit 32 applies the variation ratio Φ calculated based on the detection signal P to Mathematical Expression (5), the degree S of oxygen saturation can be calculated.

$$S = \frac{{ΦE}_{d} [ {λ}_{1} ] - {E}_{o} [ {λ}_{1} ]}{Φ ( {E}_{d} [ {λ}_{1} ] - {E}_{o} [ {λ}_{1} ] + {E}_{o} [ {λ}_{2} ] - {E}_{d} [ {λ}_{2} ]} \qquad ( 5 )$$

When Mathematical Expression (5) is derived from Mathematical Expression (4), it is assumed that the propagation range B1 of emission light from the light emission unit E1 and the propagation range B2 of emission light from the light emission unit E2 are common. In the transmissive optical sensor, as described above, since the emission light from the light emission unit E1 and the emission light from the light emission unit E2 are propagated through paths approaching each other inside the measurement site M, the above-described assumption is appropriately established. However, in the reflection-type optical sensor, in a case where the propagation range B1 and the propagation range B2 are actually different from each other, the above-described assumption is not validly established. Therefore, it is difficult to calculate the degree S of oxygen saturation with high accuracy through Mathematical Expression (5).

In each of the embodiments described above, since the propagation range B1 of emission light from the light emission unit E1 and the propagation range B2 of emission light from the light emission unit E2 can approach or overlap each other, the assumption when Mathematical Expression (5) is derived from Mathematical Expression (4) is valid. Therefore, in despite of the reflection-type optical sensor, there is an advantage in that the degree S of oxygen saturation can be calculated with high accuracy through an arithmetic operation of Mathematical Expression (5).

(3) In each of the embodiments described above, the detection device 26 provided with two light emission units E of the light emission unit E1 and the light emission unit E2 is exemplified. However, three or more light emission units E can be installed in the detection device 26. From the viewpoint that the propagation ranges B of emission light from each of the light emission units E approach or overlap each other, regardless of the number of the light emission units E, it is favorable to adopt a configuration in which the light emission unit E having a smaller arrival depth of emission light is disposed at a position farther from the light reception unit R0. The configuration in which three or more light emission units are installed is included within the scope of the invention regardless of the state of other light emission units as long as the requirement of the invention is satisfied when one of two specified light emission units serves as the first light emission unit and the other serves as the second light emission unit.

(4) In each of the embodiments described above, the measurement apparatus 100 which can be mounted on a wrist of the test subject is exemplified. However, the specific form (mounting position) of the measurement apparatus 100 is arbitrary. For example, an arbitrary form of the measurement apparatus 100 can be employed, such as a patch-type measurement apparatus which can be attached to the body of the test subject, an earring-type measurement apparatus which can be mounted on the auricle of the test subject, a finger mounted-type measurement apparatus which can be mounted on the fingertip of the test subject (for example, nail mounted-type measurement apparatus), and a head mounted-type measurement apparatus which can be mounted on the head of the test subject. However, for example, in a state where the finger mounted-type measurement apparatus 100 is mounted, a possibility of the presence of hindrance to daily life is postulated. Therefore, from the viewpoint of regularly measuring the degree S of oxygen saturation without hindrance to daily life, it is particularly favorable to adopt the measurement apparatus 100 of each of the embodiments described above which can be mounted on the wrist of the test subject. The measurement apparatus 100 in a form of being mounted in various types of electronic equipment such as a wristwatch (for example, externally attached) can be realized.

(5) In each of the embodiments described above, the degree S of oxygen saturation is measured. However, the type of biological information is not limited to the exemplification above. For example, it is possible to employ a configuration in which the pulse, the blood flow velocity, and the blood pressure are measured as the biological information, and a configuration in which the blood component concentration such as the blood glucose concentration, the hemoglobin concentration, the blood oxygen concentration, and the neutral fat concentration is measured as the biological information. In the configuration in which the blood flow velocity is measured as the biological information, a laser irradiator emitting coherent laser light which has a narrow bandwidth and is emitted via resonance of a resonator is favorably utilized as the light emission unit E.

The entire disclosure of Japanese Patent Application No. 2016-042293 is hereby incorporated herein by reference.

What is claimed is:

1. A measurement apparatus comprising:
a first light emission unit that emits light having a first wavelength;
a second light emission unit that emits light having a second wavelength of which an arrival depth with respect to a measurement site exceeds the arrival depth of the light having the first wavelength;
a light reception unit that generates a detection signal corresponding to a light reception level of light arriving from the measurement site; and
an analysis unit that acquires biological information corresponding to the detection signal,
wherein the first light emission unit, the second light emission unit, and the light reception unit are installed on a detection surface facing the measurement site, and a distance between the first light emission unit and the light reception unit exceeds a distance between the second light emission unit and the light reception unit.

2. The measurement apparatus according to claim 1,
wherein the first light emission unit, the second light emission unit, and the light reception unit are collinearly positioned.

3. The measurement apparatus according to claim 1,
wherein the light reception unit includes a first light reception unit receiving light which is emitted from the first light emission unit and passes through the measurement site, and a second light reception unit receiving light which is emitted from the second light emission unit and passes through the measurement site, and
wherein a distance between the first light emission unit and the first light reception unit exceeds a distance between the second light emission unit and the second light reception unit.

4. The measurement apparatus according to claim 3,
wherein the first light emission unit, the second light emission unit, the first light reception unit, and the second light reception unit are collinearly positioned.

5. The measurement apparatus according to claim 4,
wherein the first light emission unit and the first light reception unit are positioned between the second light emission unit and the second light reception unit.

6. The measurement apparatus according to claim 3
wherein a straight line passing through the first light emission unit and the first light reception unit and a straight line passing through the second light emission unit and the second light reception unit intersect each other.

7. The measurement apparatus according to claim 1,
wherein the light having the first wavelength is near infrared light, and
wherein the light having the second wavelength is red light.

8. The measurement apparatus according to claim 1,
wherein the light having the first wavelength is green light, and
wherein the light having the second wavelength is near infrared light or red light.

9. A detection device which generates a detection signal used for generating biological information, the detection device comprising:
a first light emission unit that emits light having a first wavelength;
a second light emission unit that emits light having a second wavelength of which an arrival depth with respect to a measurement site exceeds the arrival depth of the light having the first wavelength; and
a light reception unit that generates a detection signal corresponding to a light reception level of light arriving from the measurement site,
wherein the first light emission unit, the second light emission unit, and the light reception unit are installed on a detection surface facing the measurement site, and a distance between the first light emission unit and the light reception unit exceeds a distance between the second light emission unit and the light reception unit.
